(19) United States
(12) Patent Application Publication
(10) Pub. No.: US 2026/0241048 A1
(43) Pub. Date: Aug. 20, 2026

(54) PSMA-TARGETED RADIOPHARMACEUTICALS AND DNA DAMAGE RESPONSE INHIBITOR COMBINATION THERAPY

(71) Applicant: FUSION PHARMACEUTICALS INC., Hamilton (CA)

(72) Inventors: John R. FORBES, Burlington (CA); Brigitte Lise THÉRIAULT, Whitby (CA); Shekoufeh ALMASI, Oakville (CA); Vishal Subramony GAYATHRI, Milton (CA)

(21) Appl. No.: 19/474,981

(22) PCT Filed: Apr. 12, 2024
(86) PCT No.: PCT/CA2024/050466

§ 371 Date: Oct. 13, 2025

Related U.S. Application Data
(60) Provisional application No. 63/496,279, filed on Apr. 14, 2023.

(51) Int. Cl. A61K 51/04; A61K 31/4745; A61K 31/496; A61K 31/502; A61K 31/522; A61K 31/5377; A61P 35/00

(52) U.S. Cl. CPC A61K 51/0497; A61K 31/4745; A61K 31/496; A61K 31/502; A61K 31/522; A61K 31/5377; A61K 51/044; A61P 35/00; A61K 2121/00

(57) ABSTRACT

Combination therapies comprising administering PSMA-targeted radiopharmaceuticals and DNA damage response inhibitors.

### CROSS-REFERENCE TO RELATED APPLICATIONS

This application claims the benefit of, and priority to, U.S. Ser. No. 63/496,279, filed Apr. 14, 2023, the contents of which are incorporated herein by reference.

### BACKGROUND

DNA single stranded breaks and double stranded breaks occur for a variety of reasons, including cellular exposure to exogenous sources of DNA damaging agents such as radiopharmaceuticals or due to genetic mutations in the pathways that include the BRCA, PTEN and ATR proteins. Such DNA breaks are repaired through multiple pathways, and inhibition of these repair pathways via administration of DNA Damage Repair inhibitors (DDRis) results in an accumulation of single and/or double stranded breaks. DDRis have been investigated as cancer therapeutics based on this mechanism, as increased DNA damage leads to increased cell death rates in cancer cells. Examples of DDRis include PARP inhibitors (PARPis), ATM inhibitors (ATMis), ATR inhibitors (ATRis), and DNA-PK inhibitors (DNA-PKis).

However, the mutations that enable DDRi monotherapy in cancer cells can also be found in non-cancerous somatic cells, which can result in undesirable normal tissue toxicities for these treatments. Further, many DDRis have exhibited only modest efficacy in vivo when used as monotherapies, and their use may be restricted to cancer types that are already deficient in some aspect of DNA repair capacity (e.g., PARPi for treatment of BRCA1/2 deficient cancers).

Therefore, there is a need for improved treatments of cancer. In particular, there is a need for increases in efficacy, which do not enhance toxicity in the patient.

### SUMMARY

The present disclosure encompasses the insight that inhibition of DNA damage repair mechanisms used in combination with a therapy that targets DNA breaks specifically to cancer cells (and not normal tissues) may lead to better therapies with improved efficacy. Radioactive decay can cause direct physical damage (such as single or double-stranded DNA breaks) or indirect damage (such as by-stander or crossfire effects) to the biomolecules that constitute a cell. Radiopharmaceuticals are drugs that deliver radioisotopes to cancer cells and thereby provide a mechanism to generate DNA damage with anti-cancer therapeutic effect. The present disclosure provides the combination of ²²⁵Ac-radiopharmaceuticals, specifically small molecule-based radiopharmaceuticals targeting Prostate Specific Membrane Antigen (PSMA) positive tumors labeled with actinium-225, with DDRis to treat or ameliorate cancer.

More specifically, provided are methods of treating a mammal having cancers expressing Prostate Specific Membrane Antigen (PSMA), said method comprising:
(i) administering to the mammal an ²²⁵Ac-radiopharmaceutical, wherein the mammal has received or is receiving a DNA damage response inhibitor (DDRi);
(ii) administering to a mammal a DDRi, wherein the mammal has received or is receiving an ²²⁵Ac-radiopharmaceutical; or
(iii) administering to a mammal a DDRi at the same time as administering to the mammal an ²²⁵Ac-radiopharmaceutical, wherein in each occurrence said ²²⁵Ac-radiopharmaceutical comprises ²²⁵Ac chelated with a compound of Formula I, or a stereoisomer thereof:

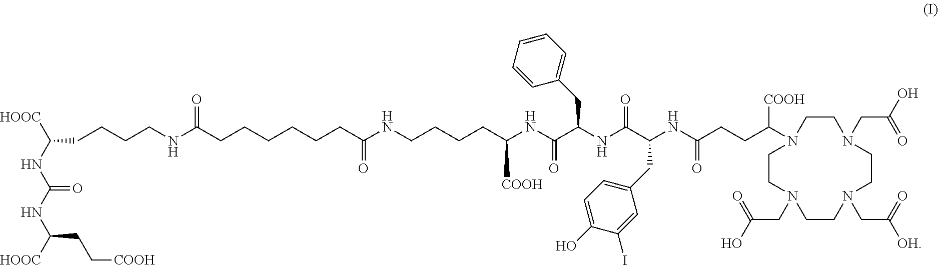 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

In some embodiments, said method comprises administering to a mammal a DDRi, wherein the mammal has received or is receiving an ²²⁵Ac-radiopharmaceutical.

In some embodiments, said ²²⁵Ac-radiopharmaceutical comprises ²²⁵Ac chelated with the following structure:

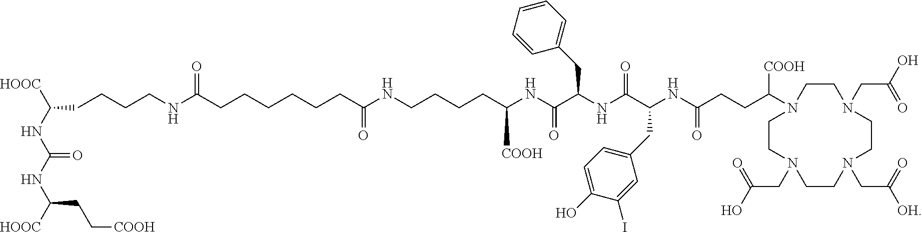 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

In some embodiments, the DDRi is a PARP inhibitor. In certain embodiments, the PARP inhibitor is a small molecule PARP inhibitor. In certain embodiments, the small molecule PARP inhibitor is selected from the group consisting of niparib, niraparib, olaparib, saruparib, talazoparib, pamiparib, rucaparib (camsylate), and veliparib, or an analog thereof. In certain embodiments, the small molecule PARP inhibitor is olaparib.

In some embodiments, the DDRi is an ATR or ATM inhibitor. In certain embodiments, the ATR or ATM inhibitor is a small molecule ATR or ATM inhibitor. In certain embodiments, the small molecule ATR or ATM inhibitor is selected from the group consisting of AZ20, AZD0156, AZD1390, AZD6738, BAY-1895344 (also known as elimusertib), EPT-46464, M3541, M4344, M6620 (formerly known as VE-922 or VX-970), NU6027, and VE-821, or an analog thereof. In certain embodiments, the small molecule ATR or ATM inhibitor is AZD1390, BAY-1895344 (also known as elimusertib), or an analog thereof.

In some embodiments, the DDRi is a DNA-protein kinase (DNA-PK) inhibitor, a WEE1 inhibitor, a Chk1 inhibitor, or a Chk2 inhibitor. In certain embodiments, the DDRi is a DNA-PK inhibitor selected from the group consisting of AZD7648, KU-0060648, NU7026, NU7441 (KU-57788), PI-103, PIK-75 HCI, PP121, and SF2523, or an analog thereof. In certain embodiments, the DNA-PK inhibitor is AZD7648 or an analog thereof.

In some embodiments, the mammal is a human.

In some embodiments, said ²²⁵Ac-radiopharmaceutical is administered at a dosage of less than 1 MBq/kg of body weight of said mammal.

In some embodiments, said ²²⁵Ac-radiopharmaceutical is administered at a dosage of less than 250 kBq/kg of body weight of said mammal.

In some embodiments, said ²²⁵Ac-radiopharmaceutical is administered at a dosage of less than 100 kBq/kg of body weight of said mammal.

In some embodiments, said ²²⁵Ac-radiopharmaceutical is administered as a unitary dosage of less than 15 MBq to said mammal.

In some embodiments, said ²²⁵Ac-radiopharmaceutical is administered as a unitary dosage of less than 10 MBq to said mammal.

In some embodiments, said ²²⁵Ac-radiopharmaceutical is administered as a unitary dosage of less than 5 MBq to said mammal.

In some embodiments, the cancer is selected from the group consisting of colorectal cancer, ductal pancreatic adenocarcinoma, small cell lung cancer, prostate cancer, breast cancer, meningioma, Ewing's sarcoma, pleural mesothelioma, head and neck cancer, non-small cell lung cancer, gastrointestinal stromal tumors, uterine leiomyoma, and cutaneous T-cell lymphoma.

In some embodiments, said cancer is colorectal cancer or ductal pancreatic adenocarcinoma.

In some embodiments, said administration results in a decrease in tumor volume, a stable tumor volume, or a reduced rate of increase in tumor volume.

In some embodiments, said administration results in a decreased incidence of recurrence or metastasis.

### BRIEF DESCRIPTION OF THE DRAWINGS

FIG. 1 is a schematic demonstrating the dosing schedule for olaparib (PARP inhibitor) in a PC3-PSMA xenograft model.

FIG. 3 is a schematic demonstrating the dosing schedule for elimusertib (also known as BAY-1895344, an ATR inhibitor) treatment in a PC3-PSMA xenograft model.

FIG. 2A, FIG. 2B, and FIG. 2C illustrate relative tumor volume and overall survival, respectively, in a PC3-PSMA xenograft model after treatment with olaparib

FIG. 4A, FIG. 4B, and FIG. 4C illustrate relative tumor volume and overall survival, respectively, in a PC3-PSMA xenograft model after treatment with elimusertib (also known as BAY-1895344).

### DETAILED DESCRIPTION

The present disclosure relates to combination therapies for treating or ameliorating cancer using certain radiopharmaceuticals and DNA damage response inhibitors in combination. In particular, the radiopharmaceuticals are ²²⁵Ac-chelated small molecules targeting Prostate Specific Membrane Antigen (PSMA).

Radio-labelled targeting moieties (also known as radiopharmaceuticals) are designed to target a protein or receptor (e.g., PSMA) that is upregulated in a disease state and/or specific to diseased cells (e.g., tumor cells) to deliver a radioactive payload to damage and kill cells of interest.

### Definitions

### Chemical Terms

The term “isomer,” as used herein, means any tautomer, stereoisomer, enantiomer, or diastereomer of any compound. It is recognized that the compound of Formula I has one or more chiral centers and, therefore, can exist as stereoisomers, such as diastereomers (e.g., enantiomers (i.e., (+) or (−))). Unless otherwise noted, chemical structures depicted herein encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds may exist in different tautomeric forms, all of the latter being included within the scope of the present disclosure.

The term “diastereomer,” as used herein means stereoisomers that are not mirror images of one another and are non-superimposable on one another.

The term “enantiomer,” as used herein, means each individual optically active form of a compound, having an optical purity or enantiomeric excess (as determined by methods standard in the art) of at least 80% (i.e., at least 90% of one enantiomer and at most 10% of the other enantiomer), preferably at least 90% and more preferably at least 98%.

### Biological Terms

ATM stands for ataxia-telangiectasia mutated
ATR stands for ataxia telangiectasia and Rad3-related
BRCA stands for breast cancer gene
Chk1 stands for checkpoint kinase 1
Chk2 stands for checkpoint kinase 2
DDR stands for DNA damage response
DNA stands for deoxyribonucleic acid
DNA-PK stands for DNA-dependent protein kinase
NTSR1 stands for neurotensin receptor 1
PARP stands for poly-ADP ribose polymerase
PTEN stands for phosphatase and tensin homolog deleted on chromosome 10
WEE1 represents WEE1 G2 checkpoint kinase

### Other Terms

As used herein, the term “about” or “approximately” refers to a ±10% variation from the recited quantitative value (and includes the recited quantitative value itself) unless otherwise indicated or inferred from the context. For example, unless otherwise stated or inferred from the context, a dose of about 100 kBq/kg indicates a dose range of 100±10% kBq/kg, i.e., from 90 kBq/kg to 110 kBq/kg, inclusive.

As used herein, the term “administered in combination,” “combined administration,” or “co-administered” means that two or more agents are administered to a subject at the same time or within an interval such that there may be an overlap of an effect of each agent on the patient. Thus, two or more agents that are administered in combination need not be administered together. In some embodiments, they are administered within 90 days (e.g., within 80, 70, 60, 50, 40, 30, 20, 10, 5, 4, 3, 2, or 1 day(s)), within 28 days (e.g., with 14, 7, 6, 5, 4, 3, 2, or 1 day(s), within 24 hours (e.g., 12, 6, 5, 4, 3, 2, or 1 hour(s), or within about 60, 30, 15, 10, 5, or 1 minute of one another. In some embodiments, the administrations of the agents are spaced sufficiently closely together such that a combinatorial effect is achieved.

As used herein, “administering” an agent to a subject includes contacting cells of said subject with the agent.

The term “cancer” refers to any cancer caused by the proliferation of malignant neoplastic cells, such as tumors, neoplasms, carcinomas, sarcomas, leukemias, and lymphomas. A “solid tumor cancer” is a cancer comprising an abnormal mass of tissue, e.g., sarcomas, carcinomas, and lymphomas. A “hematological cancer” or “liquid cancer,” as used interchangeably herein, is a cancer present in a body fluid, e.g., lymphomas and leukemias.

The term “chelate” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains a therapeutic moiety or a targeting moiety.

As used herein, the term “compound,” is meant to include all stereoisomers, geometric isomers, and tautomers of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

At various places in the present specification, substituents of compounds of the present disclosure are disclosed in groups or in ranges. It is specifically intended that the present disclosure include each and every individual sub-combination of the members of such groups and ranges. For example, the term “C₁₋₆ alkyl” is specifically intended to individually disclose methyl, ethyl, C₃ alkyl, C₄ alkyl, C₅ alkyl, and C₆ alkyl. Herein a phrase of the form “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g., alkyl) per se is optional.

As used herein, the terms “decrease,” “decreased,” “increase,” “increased,” or “reduction,” “reduced,” (e.g., in reference to therapeutic outcomes or effects) have meanings relative to a reference level. In some embodiments, the reference level is a level as determined by the use of said method with a control in an experimental animal model or clinical trial. In some embodiments, the reference level is a level in the same subject before or at the beginning of treatment. In some embodiments, the reference level is the average level in a population not being treated by said method of treatment.

The term an “effective amount” of an agent (e.g., any of the foregoing conjugates), as used herein, is that amount sufficient to effect beneficial or desired results, such as clinical results, and, as such, an “effective amount” depends upon the context in which it is being applied.

The term “lower effective dose,” when used as a term in conjunction with an agent (e.g., a therapeutic agent) refers to a dosage of the agent which is effective therapeutically in the combination therapies of the present disclosure and which is lower than the dose which has been determined to be effective therapeutically when the agent is used as a monotherapy in reference experiments or by virtue of other therapeutic guidance.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers to any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts of the compounds described here that are, within the scope of sound medical judgment, suitable for use in contact with the tissues of humans and animals without undue toxicity, irritation, or allergic response. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C.G. Wermuth), Wiley-VCH, 2008. The salts can be prepared in situ during the final isolation and purification of the compounds described herein or separately by reacting the free base group with a suitable organic acid.

Compounds may have ionizable groups so as to be capable of preparation as pharmaceutically acceptable salts. These salts may be acid addition salts involving inorganic or organic acids or the salts may, in the case of acidic forms of compounds, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, or various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “radiopharmaceutical” or “radioconjugate,” as used herein, refers to any compound or conjugate that includes a radioisotope or radionuclide, such as any of the radioisotopes or radionuclides described herein.

As used herein, the term “radionuclide,” refers to an atom capable of undergoing radioactive decay (e.g., ³H, ¹⁴C, ¹⁵N, ¹⁸F, ³⁵S, ⁴⁷Sc, ⁵⁵Co, ⁶⁰Cu, ⁶¹Cu, ⁶²Cu, ⁶⁴Cu, ⁶⁷Cu, ⁷⁵Br, ⁷⁶Br, ⁷⁷Br, ⁸⁹Zr, ⁸⁶Y, ⁸⁷Y, ⁹⁰Y, ⁹⁷Ru, ⁹⁹Tc, ^{99m}Tc, ¹⁰⁵Rh, ¹⁰⁹Pd, ¹¹¹In, ¹²³I, ¹²⁴I, ¹²⁵I, ¹³¹I, ¹³⁴Ce, ¹⁴⁹Pm, ¹⁴⁹Tb, ¹⁵³Sm, ¹⁶⁶Ho, ¹⁷⁷Lu, ¹⁸⁶Re, ¹⁸⁸Re, ¹⁹⁸Au, ¹⁹⁹Au, ²⁰³Pb, ²¹¹At, ²¹²Pb, ²¹²Bi, ²¹³Bi, ²²³Ra, ²²⁵Ac, ^{227Th}, ^{229Th}, ⁶⁶Ga, ⁶⁷Ga, ⁶⁸Ga, ⁸²Rb, ^{117m}Sn, ²⁰¹Tl). The terms radioactive nuclide, radioisotope, or, radioactive isotope may also be used to describe a radionuclide. Radionuclides may be used as detection agents. In some embodiments, the radionuclide is an alpha-emitting radionuclide. Exemplary radionuclides that may be used in the present disclosure include, but are not limited to, ⁶⁴Cu, ⁶⁷Cu, ⁶⁸Ga, ⁹⁰Y, ¹⁴⁹Tb, ¹⁵³Sm, ¹⁷⁷Lu, ²¹¹At, ²¹²Bi, ²¹²Pb, ²¹³Bi, ²²³Ra, ²²⁵Ac, and ^{227Th}.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. In the context of cancer treatment, “ameliorating” may include, for example, reducing incidence of metastases, reducing tumor volume, reducing tumor vascularization and/or reducing the rate of tumor growth. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### DNA Damage and Repair Inhibitors (DDRi)

As disclosed herein, the terms “DNA Damage Response inhibitor” and “DNA Damage and Repair inhibitor” are used interchangeably. In various embodiments, a DNA Damage and Repair inhibitor (DDRi) is co-administered with a radiopharmaceutical.

DNA repair involves multiple molecular pathways that repair DNA single strand breaks (e.g., the PARP pathway) and double stranded breaks (e.g., BRCA and other genes such as ATR/ATM). PARP inhibition (PARPi) results in a failure of single stranded break repair, which further leads to double stranded breaks. Available PARP inhibitors act through both PARP enzyme inhibition and DNA-trapping. Tumor cells with BRCA and/or PTEN mutations are sensitive to PARPi. ATR inhibition (ATRi) results in a failure to repair double stranded breaks—the accumulation of double stranded breaks results in cell death. These inhibitors act by preventing homologous recombination and non-homologous end joining mechanisms.

The present disclosure relates to combination therapy with radiopharmaceuticals and DNA Damage and Repair inhibitors. It has been found that this type of combination therapy results in unexpected improvement in the treatment of cancer, especially in cancers that would not be expected to be responsive to the DDRi.

In some embodiments, the DDRi is a PARP inhibitor (PARPi). In some embodiments, the PARPi is selected from the group consisting of niparib, niraparib, olaparib, saruparib, pamiparib, rucaparib (camsylate), talazoparib, and veliparib, or an analog thereof. In some embodiments, the PARPi is adavosertib, AZD2811, or an analog thereof.

In some embodiments, the DDRi is an ATM/ATR inhibitor. In some embodiments the ATM/ATR inhibitor is selected from the group consisting of AZ20, AZD0156, AZD1390, AZD6738, BAY-1895344 (also known as elimusertib), EPT-46464, M3541, M4344, M6620 (formerly known as VE-922 or VX-970), NU6027, and VE-821, or an analog thereof. In certain embodiments, the ATM/ATR inhibitor is AZD1390 or an analog thereof.

In some embodiments, the DDRi is a WEE1 inhibitor, a Chk1 inhibitor, or a Chk2 inhibitor. Examples of a WEE1 inhibitor, a Chk1 inhibitor, or a Chk2 inhibitor include those known in the field.

In some embodiments, the DDRi is a DNA-dependent protein kinase (DNA-PK) inhibitor. Non-limiting examples of DNA-PK inhibitors include, but are not limited to, AZD7648, KU-0060648, NU7026, NU7441 (KU-57788), PI-103, PIK-75 HCI, PP121, SF2523, and analogs thereof. In certain embodiments, the DNA-PK inhibitor is AZD7648 or an analog thereof.

### Subjects

In some disclosed methods, a therapy (e.g., comprising a therapeutic agent) is administered to a subject. In some embodiments, the subject is a mammal, e.g., a human.

In some embodiments, the subject has received or is receiving another therapy. For example, in some embodiments, the subject has received or is receiving a radiopharmaceutical. In some embodiments, the subject has received or is receiving a DDRi.

In some embodiments, the subject has cancer or is at risk of developing cancer. For example, the subject may have been diagnosed with cancer. The cancer may be a primary cancer or a metastatic cancer. Subjects may have any stage of cancer, e.g., stage I, stage II, stage III, or stage IV with or without lymph node involvement and with or without metastases. Provided compositions may prevent or reduce further growth of the cancer and/or otherwise ameliorate the cancer (e.g., prevent or reduce metastases). In some embodiments, the subject does not have cancer but has been determined to be at risk of developing cancer, e.g., because of the presence of one or more risk factors such as environmental exposure, presence of one or more genetic mutations or variants, family history, etc. In some embodiments, the subject has not been diagnosed with cancer.

In some embodiments, the cancer is a solid tumor.

In some embodiments, the solid tumor cancer is breast cancer, non-small cell lung cancer, small cell lung cancer, pancreatic cancer, head and neck cancer, prostate cancer, colorectal cancer, sarcoma, adrenocortical carcinoma, neuroendocrine cancer, Ewing's Sarcoma, multiple myeloma, or acute myeloid leukemia.

In some embodiments, the cancer is a non-solid (e.g., liquid (e.g., hematologic)) cancer.

### Administration and Dosage

### Effective and Lower Effective Doses

The present disclosure provides combination therapies in which the amounts of each therapeutic may or may not be, on their own, therapeutically effective. For example, provided are methods comprising administering a first therapy and a second therapy in amounts that together are effective to treat or ameliorate a disorder, e.g., cancer. In some embodiments, at least one of the first and second therapies is administered to the subject in a lower effective dose. In some embodiments, both the first and the second therapies are administered in lower effective doses.

In some embodiments, the first therapy comprises a radiopharmaceutical and the second therapy comprises a DDRi.

In embodiments, the radiopharmaceutical is a ²²⁵Ac-radiopharmaceutical that comprises ²²⁵Ac chelated with a compound having the following structure:

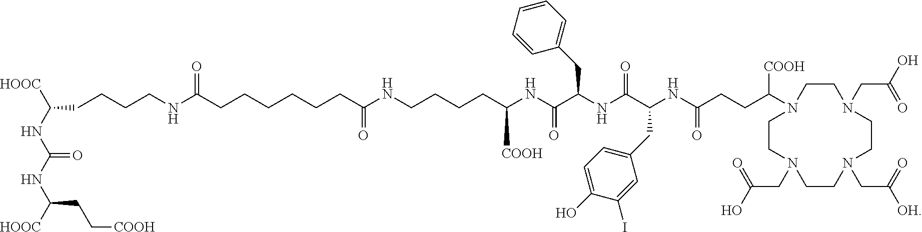 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

In some embodiments, the first therapy comprises a DDRi and the second therapy comprises a radiopharmaceutical.

In some embodiments, therapeutic combinations as disclosed herein are administered to a subject in a manner (e.g., dosing amount and timing) sufficient to cure or at least partially arrest the symptoms of the disorder and its complications. In the context of a single therapy (a “monotherapy”), an amount adequate to accomplish this purpose is defined as a “therapeutically effective amount,” an amount of a compound sufficient to substantially improve at least one symptom associated with the disease or a medical condition. The “therapeutically effective amount” typically varies depending on the therapeutic. For known therapeutic agents, the relevant therapeutically effective amounts may be known to or readily determined by those of skill in the art.

For example, in the treatment of cancer, an agent or compound that decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual. For example, a treatment may be therapeutically effective if it causes a cancer to regress or to slow the cancer's growth.

The dosage regimen (e.g., amounts of each therapeutic, relative timing of therapies, etc.) that is effective for these uses may depend on the severity of the disease or condition and the weight and general state of the subject. For example, the therapeutically effective amount of a particular composition comprising a therapeutic agent applied to mammals (e.g., humans) can be determined by the person of ordinary skill in the art with consideration of individual differences in age, weight, and the condition of the mammal. Because certain conjugates of the present disclosure exhibit an enhanced ability to target cancer cells and residualize, the dosage of these compounds can be lower than (e.g., less than or equal to about 90%, 75%, 50%, 40%, 30%, 20%, 15%, 12%, 10%, 8%, 7%, 6%, 5%, 4%, 3%, 2%, 1%, 0.5%, or 0.1% of) the equivalent dose of required for a therapeutic effect of the unconjugated agent. Therapeutically effective and/or optimal amounts can also be determined empirically by those of skill in the art. Thus, lower effective doses can also be determined by those of skill in the art.

Single or multiple administrations of a radiopharmaceutical or a composition (e.g., a pharmaceutical composition comprising a therapeutic agent or a radiopharmaceutical) can be carried out with dose levels and pattern being selected by the treating physician. The dose and administration schedule can be determined and adjusted based on the severity of the disease or condition in the subject, which may be monitored throughout the course of treatment according to the methods commonly practiced by clinicians or those described herein.

In the disclosed combination therapy methods, the first and second therapies may be administered sequentially or concurrently to a subject. For example, a first composition comprising a first therapeutic agent and a second composition comprising a second therapeutic agent may be administered sequentially or concurrently to a subject. Alternatively, a composition comprising a combination of a first therapeutic agent and a second therapeutic agent may be administered to the subject.

In some embodiments, the radiopharmaceutical is administered in a single dose. In some embodiments, the radiopharmaceutical is administered more than once, i.e., multiple doses. When the radiopharmaceutical is administered more than once, the dose of each administration may be the same or different.

In some embodiments, the DDRi is administered in a single dose. In some embodiments, the DDRi is administered more than once, e.g., at least twice, at least three times, etc. In some embodiments, the DDRi is administered multiple times according to a regular or semi-regular schedule, e.g., once every approximately two weeks, once a week, twice a week, three times a week, or more than three times a week. When the DDRi is administered more than once, the dose of each administration may be the same or different. For example, the DDRi may be administered in an initial dose amount, and then subsequent dosages of the DDRi may be higher or lower than the initial dose amount.

In some embodiments, the first dose of the DDRi is administered at the same time as the first dose of the radiopharmaceutical. In some embodiments, the first dose of the DDRi is administered before the first dose of radiopharmaceutical. In some embodiments, the first dose of the DDRi is administered after the first dose of radiopharmaceutical. In some embodiments, subsequent doses of the DDRi are administered.

In some embodiments, the present disclosure provides methods comprising administering to a mammal a DDRi at a dose of about 1 to 100 mg/kg (e.g., about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, and about 100 mg/kg). In some embodiments, the present disclosure provides methods comprising administering to a mammal a DDRi at a dose of about 5 mg/kg, about 20 mg/kg, about 40 mg/kg, about 50 mg/kg, or about 100 mg/kg.

In some embodiments, the present disclosure provides methods comprising administering to a mammal an ²²⁵Ac-radiopharmaceutical at a dosage of less than 2 MBq/kg (e.g., less than 1.5 MBq/kg, less than 1 MBq/kg, less than 500 kBq/kg, less than 400 kBq/kg, less than 300 kBq/kg, less than 250 kBq/kg, less than 200 kBq/kg, less than 150 kBq/kg, less than 100 kBq/kg, or less than 50 kBq/kg) of body weight of said mammal. Each of the dose may be administered multiple times to the mammal.

In some embodiments, the present disclosure provides methods comprising administering to a mammal an ²²⁵Ac-radiopharmaceutical at a dosage of about 0.1 MBq/kg to about 1.0 MBq/kg (e.g., 0.1 MBq/kg, 0.2 MBq/kg, 0.3 MBq/kg, 0.4 MBq/kg, 0.5 MBq/kg, 0.6 MBq/kg, 0.7 MBq/kg, 0.8 MBq/kg, 0.9 MBq/kg, and 1.0 MBq/kg,) of body weight of said mammal. In some embodiments, the present disclosure provides methods comprising administering to a mammal an ²²⁵Ac-radiopharmaceutical at a dosage of about 0.5 MBq/kg of body weight of said mammal.

In some embodiments, the present disclosure provides methods comprising administering to a mammal an ²²⁵Ac-radiopharmaceutical at a dosage of about 1 MBq/kg to about 10 MBq/kg of body weight of said mammal. In some embodiments, the present disclosure provides methods comprising administering to a mammal an ²²⁵Ac-radiopharmaceutical at a dosage of about 1 MBq/kg to about 5 MBq/kg of body weight of said mammal. In some embodiments, the present disclosure provides methods comprising administering to a mammal an ²²⁵Ac-radiopharmaceutical at a dosage of about 1 MBq/kg to about 2 MBq/kg of body weight of said mammal. Each of the dose may be administered one time or multiple times to the mammal. In some embodiments, the present disclosure provides methods comprising administering to a mammal an ²²⁵Ac-radiopharmaceutical at a dosage of about 1 MBq/kg of body weight of said mammal. Each of the dose may be administered one time or multiple times to the mammal.

In some embodiments, said ²²⁵Ac-radiopharmaceutical is administered at a dosage of less than 250 kBq/kg (e.g., about 240 kBq/kg, about 220 kBq/kg, about 200 kBq/kg, about 180 kBq/kg, about 160 kBq/kg, about 150 kBq/kg, about 140 kBq/kg, about 130 kBq/kg, about 120 kBq/kg, about 110 kBq/kg, or about 100 kBq/kg) of body weight of said mammal. Each of the dose may be administered multiple times to the mammal.

In some embodiments, said ²²⁵Ac-radiopharmaceutical is administered at a dosage of less than 100 kBq/kg (e.g., about 90 kBq/kg, about 80 kBq/kg, about 70 kBq/kg, about 60 kBq/kg, about 50 kBq/kg, about 40 kBq/kg, about 30 kBq/kg, about 20 kBq/kg, or about 10 kBq/kg) of body weight of said mammal. Each of the dose may be administered multiple times to the mammal.

In some embodiments, said ²²⁵Ac-radiopharmaceutical is administered as a unitary dosage of less than 15 MBq (e.g., about 14 MBq, about 13 MBq, about 12 MBq, about 11 MBq, about 10 MBq, about 9 MBq, about 8 MBq, about 7 MBq, about 6 MBq, about 5 MBq, about 4 MBq, about 3 MBq, about 2 MBq, about 1 MBq) to said mammal. Each of the unitary dosage may be administered multiple times to the mammal.

In some embodiments, said ²²⁵Ac-radiopharmaceutical is administered as a unitary dosage of less than 10 MBq to said mammal. Each of the unitary dosage may be administered multiple times to the mammal.

In some embodiments, said ²²⁵Ac-radiopharmaceutical is administered as a unitary dosage of less than 5 MBq to said mammal. Each of the unitary dosage may be administered multiple times to the mammal.

In some embodiments, radiopharmaceuticals (or a composition thereof) and DDRis (or a composition thereof) are administered within 28 days (e.g., within 14, 7, 6, 5, 4, 3, 2, or 1 day(s)) of each other.

In some embodiments, radiopharmaceuticals (or a composition thereof) and DDRis (or a composition thereof) are administered within 90 days (e.g., within 80, 70, 60, 50, 40, 30, 20, 10, 5, 4, 3, 2, or 1 day(s)) of each other. In various embodiments, the DDRi is administered at the same time as radiopharmaceutical. In various embodiments, the DDRi is administered multiple times after the first administration of radiopharmaceutical.

In some embodiments, compositions (such as compositions comprising radiopharmaceuticals) are administered for radiation treatment planning or diagnostic purposes. When administered for radiation treatment planning or diagnostic purposes, compositions may be administered to a subject in a diagnostically effective dose and/or an amount effective to determine the therapeutically effective dose. In some embodiments, a first dose of disclosed conjugate or a composition (e.g., pharmaceutical composition) thereof is administered in an amount effective for radiation treatment planning, followed administration of a combination therapy including a conjugate as disclosed herein and another therapeutic.

Pharmaceutical compositions comprising one or more agents (e.g., radiopharmaceuticals and/or DDRis) can be formulated for use in accordance with disclosed methods and systems in a variety of drug delivery systems. One or more physiologically acceptable excipients or carriers can also be included in the composition for proper formulation. Examples of suitable formulations are found in Remington's Pharmaceutical Sciences, Mack Publishing Company, Philadelphia, PA, 17^{th} ed., 1985. For a brief review of methods for drug delivery, see, e.g., Langer (Science 249:1527-1533, 1990).

### Formulations

Pharmaceutical compositions may be formulated for any of various administration routes, including, but not limited to, parenteral, intranasal, topical, oral, or local administration, such as by a transdermal means, for prophylactic and/or therapeutic treatment. Pharmaceutical compositions can be administered parenterally (e.g., by intravenous, intramuscular, or subcutaneous injection), or by oral ingestion, or by topical application or intraarticular injection at areas affected by the vascular or cancer condition. Examples of additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Also specifically contemplated are sustained release administration, by such means as depot injections or erodible implants or components. Suitable compositions include compositions comprising include agents (e.g., compounds as disclosed herein) dissolved or suspended in an acceptable carrier, preferably an aqueous carrier, e.g., water, buffered water, saline, or PBS, among others, e.g., for parenteral administration. Compositions may contain pharmaceutically acceptable auxiliary substances to approximate physiological conditions, such as pH adjusting and buffering agents, tonicity adjusting agents, wetting agents, or detergents, among others. In some embodiments, compositions are formulated for oral delivery; for example, compositions may contain inert ingredients such as binders or fillers for the formulation of a unit dosage form, such as a tablet or a capsule. In some embodiments, compositions are formulated for local administration; for example, compositions may contain inert ingredients such as solvents or emulsifiers for the formulation of a cream, an ointment, a gel, a paste, or an eye drop.

Compositions may be sterilized, e.g., by conventional sterilization techniques, or sterile filtered. Aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 6 and 7, such as 6 to 6.5. In some embodiments, compositions in solid form are packaged in multiple single dose units, each containing a fixed amount of the above-mentioned agent or agents, such as in a sealed package of tablets or capsules. In some embodiments, compositions in solid form are packaged in a container for a flexible quantity, such as in a squeezable tube designed for a topically applicable cream or ointment.

### Effects

In some embodiments, methods of the present disclosure result in a therapeutic effect.

In some embodiments, the therapeutic effect comprises a decrease in tumor volume, a stable tumor volume, or a reduced rate of increase in tumor volume. In some embodiments, the therapeutic effect comprises a decreased incidence of recurrence or metastasis. In some embodiments, the therapeutic effect comprises tumor regression.

### OTHER AGENTS

In some embodiments, disclosed methods further include administering an antiproliferative agent, radiation sensitizer, or an immunoregulatory or immunomodulatory agent.

By “antiproliferative” or “antiproliferative agent,” as used interchangeably herein, is meant any anticancer agent, including those antiproliferative agents listed in Table 1, any of which can be used in combination with a radiopharmaceutical to treat a condition or disorder. Antiproliferative agents also include organo-platinum derivatives, naphthoquinone and benzoquinone derivatives, chrysophanic acid and anthraquinone derivatives thereof.

By “immunoregulatory agent” or “immunomodulatory agent,” as used interchangeably herein, is meant any immuno-modulator, including those listed in Table 1, any of which can be used in combination with a radiopharmaceutical provided herein.

As used herein, “radiation sensitizer” includes any agent that increases the sensitivity of cancer cells to radiation therapy. Radiation sensitizers may include, but are not limited to, 5-fluorouracil, analogs of platinum (e.g., cisplatin, carboplatin, oxaliplatin), gemcitabine, EGFR antagonists (e.g., cetuximab, gefitinib), farnesyltransferase inhibitors, COX-2 inhibitors, bFGF antagonists, and VEGF antagonists.

TABLE 1

| | | |
|---|---|---|
| Alkylating agents | Busulfan | Chlorambucil |
| | dacarbazine | procarbazine |
| | ifosfamide | altretamine |
| | hexamethylmelamine | estramustine phosphate |
| | thiotepa | mechlorethamine |
| | dacarbazine | streptozocin |
| | lomustine | temozolomide |
| | cyclophosphamide | Scmustinc |
| Platinum agents | spiroplatin | lobaplatin (Aeterna) |
| | tetraplatin | satraplatin (Johnson Matthey) |
| | ormaplatin | BBR-3464 (Hoffmann-La Roche) |
| | iproplatin | Miriplatin |
| | picoplatin | AP-5280 (Access) |
| | oxaliplatin | cisplatin |
| | carboplatin | |
| Antimetabolites | azacytidine | trimetrexate |
| | Floxuridine | deoxycoformycin |
| | 2-chlorodeoxyadenosine | pentostatin |
| | 6-mercaptopurine | hydroxyurea |
| | 6-thioguanine | decitabine (SuperGen) |
| | cytarabine | clofarabine (Bioenvision) |
| | 2-fluorodeoxy cytidine | irofulven (MGI Pharma) |
| | methotrexate | DMDC (Hoffmann-La Roche) |
| | tomudex | ethynylcytidine (Taiho) |
| | fludarabine | gemcitabine |
| | raltitrexed | capecitabine |
| Topoisomerase | amsacrine | exatecan mesylate (Daiichi) |
| inhibitors | epirubicin | quinamed (ChemGenex) |
| | etoposide | gimatecan (Sigma-Tau) |
| | teniposide or mitoxantrone | diflomotecan (Beaufour-Ipsen) |
| | 7-ethyl-10-hydroxy-camptothecin | TAS-103 (Taiho) |
| | dexrazoxanet (Topo Target) | elsamitrucin (Spectrum) |
| | pixantrone (Novuspharma) | Edotecarin |
| | rebeccamycin analogue (Exelixis) | Cositecan |
| | BBR-3576 (Novuspharma) | Belotecan |
| | rubitecan (SuperGen) | hydroxycamptothecin (SN-38) |
| | irinotecan (CPT-11) | |
| | topotecan | |
| Antitumor | valrubicin | azonafide |
| antibiotics | therarubicin | anthrapyrazole |
| | idarubicin | oxantrazole |
| | rubidazone | losoxantrone |
| | plicamycin | Sabarubicin |
| | porfiromycin | Epirubicin |
| | mitoxantrone (novantrone) | mitoxantrone |
| | amonafide | doxorubicin |
| Antimitotic | colchicine | E7010 (Abbott) |
| agents | vinblastine | PG-TXL (Cell Therapeutics) |
| | vindesine | IDN 5109 (Bayer) |
| | dolastatin 10 (NCI) | A 105972 (Abbott) |
| | rhizoxin (Fujisawa) | A 204197 (Abbott) |
| | mivobulin (Warner-Lambert) | LU 223651 (BASF) |
| | cemadotin (BASF) | D 24851 (ASTAMedica) |
| | RPR 109881A (Aventis) | ER-86526 (Eisai) |
| | TXD 258 (Aventis) | combretastatin A4 (BMS) |
| | epothilone B (Novartis) | isohomohalichondrin-B |
| | T 900607 (Tularik) | (PharmaMar) |
| | T 138067 (Tularik) | ZD 6126 (AstraZeneca) |
| | cryptophycin 52 (Eli Lilly) | AZ10992 (Asahi) |
| | vinflunine (Fabre) | IDN-5109 (Indena) |
| | auristatin PE (Teikoku Hormone) | AVLB (Prescient NeuroPharma) |
| | BMS 247550 (BMS) | azaepothilone B (BMS) |
| | BMS 184476 (BMS) | BNP-7787 (BioNumerik) |
| | BMS 188797 (BMS) | CA-4 prodrug (OXiGENE) |
| | taxoprexin (Protarga) | dolastatin-10 (NIH) |
| | SB 408075 (GlaxoSmithKline) | CA-4 (OXiGENE) |
| | Vinorelbinc | docctaxcl |
| | Trichostatin A | vincristine |
| | | paclitaxel |
| Aromatase | aminoglutethimide | YM-511 (Yamanouchi) |
| inhibitors | atamestane (BioMedicines) | formestane |
| | letrozole | exemestane |
| | anastrazole | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | edotreotide (Novartis) |
| | glufosfamide (Baxter International) | mafosfamide (Baxter International) |
| | albumin + 32P (Isotope Solutions) | apaziquone (Spectrum Pharmaceuticals) |
| | thymectacin (NewBiotics) | O6 benzyl guanine (Paligent) |
| Farnesyltransferase | arglabin (NuOncology Labs) | tipifarnib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli Lilly) |
| | tariquidar (Xenova) | biricodar dicitrate (Vertex) |
| | MS-209 (Schering AG) | |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno-modulators | interferon | dexosome therapy (Anosys) |
| | oncophage (Antigenics) | pentrix (Australian Cancer Technology) |
| | GMK (Progenics) | ISF-154 (Tragen) |
| | adenocarcinoma vaccine (Biomira) | cancer vaccine (Intercell) |
| | CTP-37 (AVI BioPharma) | norelin (Biostar) |
| | IRX-2 (Immuno-Rx) | BLP-25 (Biomira) |
| | PEP-005 (Peplin Biotech) | MGV (Progenics) |
| | synchrovax vaccines (CTL Immuno) | β-alethine (Dovetail) |
| | melanoma vaccine (CTL Immuno) | CLL therapy (Vasogen) |
| | p21 RAS vaccine (Gem Vax) | Ipilimumab (BMS), |
| | MAGE-A3 (GSK) | CM-10 (cCam Biotherapeutics) |
| | nivolumab (BMS) | atezolizumab (Genentech) |
| | abatacept (BMS) | |
| | pembrolizumab (Merck) | |
| Hormonal and | estrogens | dexamethasone |
| antihormonal agents | conjugated estrogens | prednisone |
| | ethinyl estradiol | methylprednisolone |
| | chlortrianisen | prednisolone |
| | idenestrol | aminoglutethimide |
| | hydroxyprogesterone caproate | leuprolide |
| | medroxyprogesterone | octreotide |
| | testosterone | mitotane |
| | testosterone propionate; | P-04 (Novogen) |
| | fluoxymesterone | 2-methoxyestradiol (EntreMed) |
| | methyltestosterone | arzoxifene (Eli Lilly) |
| | diethylstilbestrol | tamoxifen |
| | megestrol | toremofine |
| | bicalutamide | goserelin |
| | flutamide | Leuporelin |
| | nilutamide | bicalutamide |
| Photodynamic | talaporfin (Light Sciences) | Pd-bacteriopheophorbide (Yeda) |
| agents | Theralux (Theratechnologies) | Motexafin lutetium |
| | motexafin gadolinium | hypericin |
| | (Pharmacyclics) | |
| Kinase Inhibitors | imatinib (Novartis) | EKB-569 (Wyeth) |
| | leflunomide (Sugen/Pharmacia) | kahalide F (PharmaMar) |
| | ZD1839 (AstraZeneca) | CEP-701 (Cephalon) |
| | erlotinib (Oncogene Science) | CEP-751 (Cephalon) |
| | canertinib (Pfizer) | MLN518 (Millenium) |
| | squalaminc (Genaera) | PKC412 (Novartis) |
| | SU5416 (Pharmacia) | Phenoxodiol (Novogen) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | trastuzumab (Genentech) | Tyrphostins |
| | OSI-774 (Tarceva ™) | Gefitinib (Iressa) |
| | CI-1033 (Pfizer) | PTK787 (Novartis) |
| | SU11248 (Pharmacia) | EMD 72000 (Merck) |
| | RH3 (York Medical) | Emodin |
| | Genistein | Radicinol |
| | Radicinol | Vemurafenib (B-Raf enzyme |
| | Met-Mab (Roche) | inhibitor, Daiichi Sankyo) |

| | |
|---|---|
| SR-27897 (CCK A inhibitor, Sanofi-Synthelabo) | ceflatonin (apoptosis promotor, ChemGenex) |
| tocladesine (cyclic AMP agonist, Ribapharm) | BCX-1777 (PNP inhibitor, BioCryst) |
| alvocidib (CDK inhibitor, Aventis) | ranpirnase (ribonuclease stimulant, Alfacell) |
| CV-247 (COX-2 inhibitor, Ivy Medical) | galarubicin (RNA synthesis inhibitor, Dong-A) |
| P54 (COX-2 inhibitor, Phytopharm) | tirapazamine (reducing agent, SRI International) |
| CapCell ™ (CYP450 stimulant, Bavarian Nordic) | N-acetylcysteine (reducing agent, Zambon) |
| GCS-100 (gal3 antagonist, GlycoGenesys) | R-flurbiprofen (NF-kappaB inhibitor, Encore) |
| G17DT immunogen (gastrin inhibitor, Aphton) | 3CPA (NF-kappaB inhibitor, Active Biotech) |
| efaproxiral (oxygenator, Allos Therapeutics) | seocalcitol (vitamin D receptor agonist, Leo) |
| PI-88 (heparanase inhibitor, Progen) | 131-I-TM-601 (DNA antagonist, TransMolecular) |
| tesmilifene (histamine antagonist, YM | eflornithine (ODC inhibitor, ILEX Oncology) |
| BioSciences) | minodronic acid (osteoclast inhibitor, Yamanouchi) |
| histamine (histamine H2 receptor agonist, | indisulam (p53 stimulant, Eisai) |
| Maxim) | aplidine (PPT inhibitor, PharmaMar) |
| tiazofurin (IMPDH inhibitor, Ribapharm) | gemtuzumab (CD33 antibody, Wyeth Ayerst) |
| cilengitide (integrin antagonist, Merck KgaA) | PG2 (hematopoiesis enhancer, Pharmagenesis) |
| SR-31747 (IL-1 antagonist, Sanofi-Synthelabo) | Immunol ™ (triclosan oral rinse, Endo) |
| CCI-779 (mTOR kinase inhibitor, Wyeth) | triacetyluridine (uridinc prodrug, Wellstat) |
| exisulind (PDE V inhibitor, Cell Pathways) | SN-4071 (sarcoma agent, Signature BioScience) |
| CP-461 (PDE V inhibitor, Cell Pathways) | TransMID-107 ™ (immunotoxin, KS Biomedix) |
| AG-2037 (GARFT inhibitor, Pfizer) | PCK-3145 (apoptosis promotor, Procyon) |
| WX-UKI (plasminogen activator inhibitor, | doranidazole (apoptosis promotor, Pola) |
| Wilex) | CHS-828 (cytotoxic agent, Leo) |
| PBI-1402 (PMN stimulant, ProMetic | trans-retinoic acid (differentiator, NIH) |
| Life Sciences) | MX6 (apoptosis promotor, MAXIA) |
| bortezomib (proteasome inhibitor, Millennium) | apomine (apoptosis promotor, ILEX Oncology) |
| SRL-172 (T cell stimulant, SR Pharma) | urocidin (apoptosis promotor, Bioniche) |
| TLK-286 (glutathione S transferase inhibitor, | Ro-31-7453 (apoptosis promotor, La Roche) |
| Telik) | brostallicin (apoptosis promotor, Pharmacia) |
| PT-100 (growth factor agonist, Point | β-lapachone |
| Therapeutics) | gelonin |
| midostaurin (PKC inhibitor, Novartis) | cafestol |
| bryostatin-1 (PKC stimulant, GPC Biotech) | kahweol |
| CDA-II (apoptosis promotor, Everlife) | caffeic acid |
| SDX-101 (apoptosis promotor, Salmedix) | Tyrphostin AG |
| rituximab (CD20 antibody, Genentech | PD-1 inhibitors |
| carmustine | CTLA-4 inhibitors |
| Mitoxantrone | sorafenib |
| Bleomycin | |
| Absinthin | |
| Chrysophanic acid | |
| Cesium oxides | |
| BRAF inhibitors, | |
| PD-L1 inhibitors | |
| MEK inhibitors | |
| bevacizumab | |
| angiogenesis inhibitors | |
| dabrafenib | |

### EXAMPLES

### Example 1. Synthesis of Radiopharmaceuticals Comprising Compound of Formula I

Compounds of Formula I are small molecule antagonists targeting PSMA, which can be radiolabeled with a radionuclide such as Lutetium-177 (¹⁷⁷Lu) or Actinium-225 (225Ac) to form radionuclide-chelated radiopharmaceuticals. The synthesis of compound of Formula I, or corresponding radionuclide-chelated radiopharmaceuticals, can be referred to the following documents: Weineisen M, et al. EJNMMI Research, 2014, 4:63; Weineisen M, et al. J Nucl Med 2015, 56:1169-1176; U.S. Pat. No. 11,129,912 B1; Zacherl et al. J Nucl Med 2021, 62:669-674; and Hooijman E, et al. Pharmaceutics, 2021, 13:715.

### Example 2. Therapeutic Efficacy of a Combination of ²²⁵Ac-Compound I and Olaparib (PARP Inhibitor) Treatment in the PC3-PSMA Xenograft Model

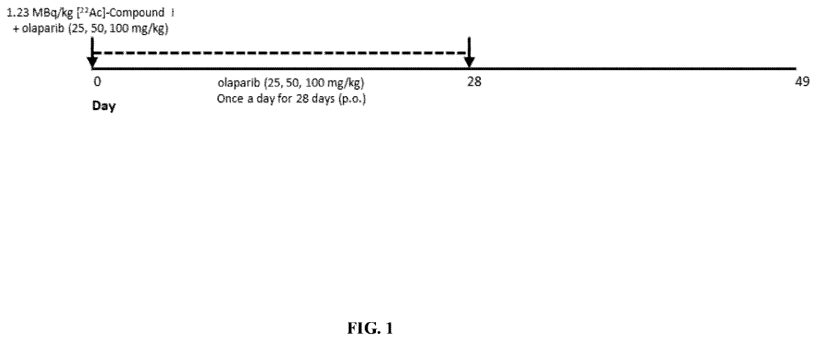

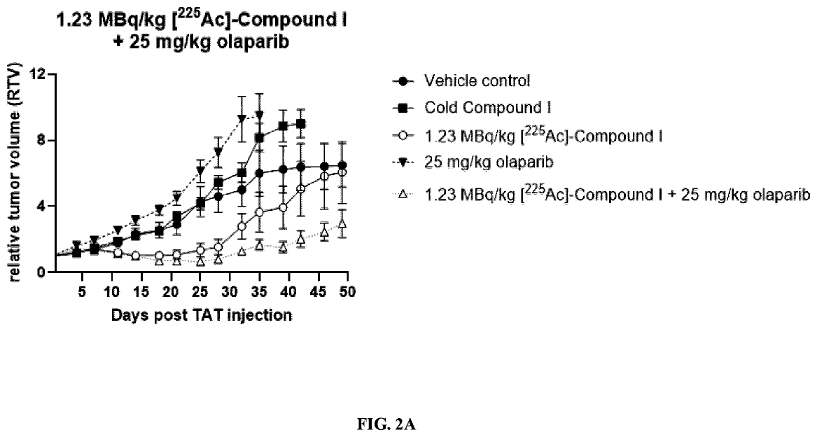

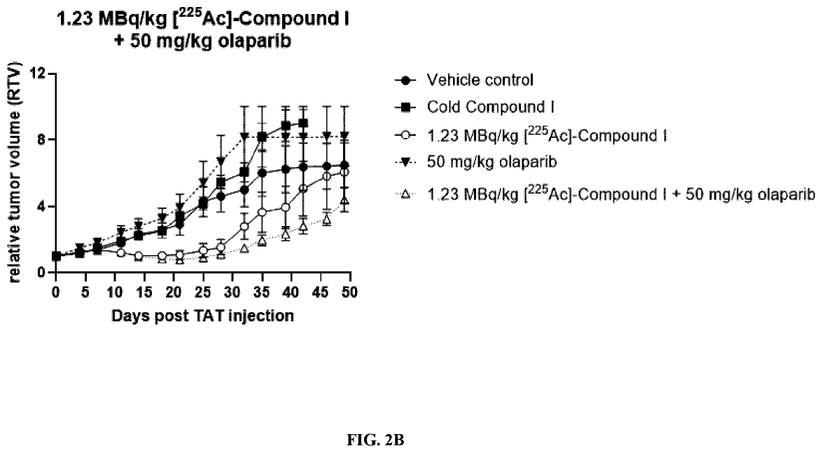

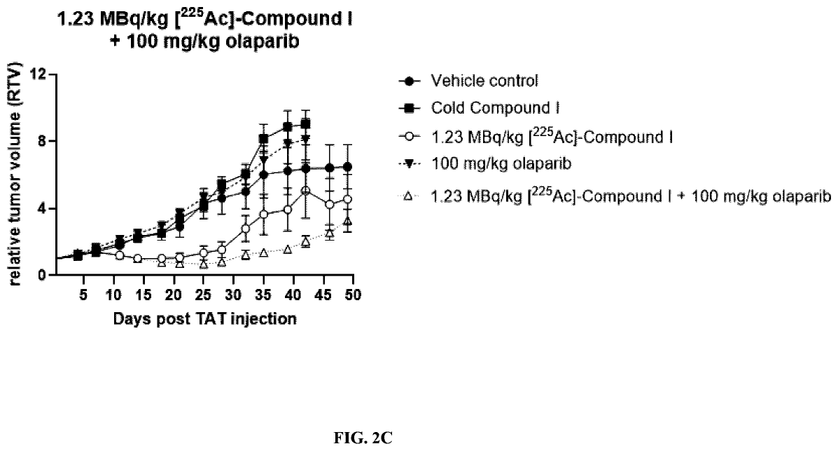

An in vivo combination study was conducted with ²²⁵Ac-Compound I and olaparib using a PSMA overexpressing PC3 (PC3-PSMA, prostate cancer) tumor xenograft bearing mouse model. Groups of mice (n=5/group) were treated with ²²⁵Ac-Compound I administered as a single dose (intravenous), at a dose sub-optimal for monotherapeutic efficacy (1.23 MBq/kg or 1 μCi, based on 30 g mice). Additional groups of mice (n=5/group) were dosed daily with olaparib (oral gavage) using a dose range of 25, 50, or 100 mg/kg for 28 consecutive days in combination with ²²⁵Ac-Compound I. Olaparib dosing started on the same day that ²²⁵Ac-Compound I was dosed (FIG. 1). Olaparib monotherapy and vehicle control groups (n=5/group) were included as controls. Tumor measurements were taken 2-3 times per week and used to calculate tumor volume. Study duration was for 50 days after initial therapeutic administration.

Tumor growth in study animals was tracked over time and expressed as Relative Tumor Volume (RTV; tumor volume on day X divided by initial tumor volume on day 0) (FIGS. 2A-2C). Single agent olaparib monotherapy was found ineffective at all dose levels tested. Olaparib combined with 1.23 MBq/kg of [²²⁵Ac]-Compound I demonstrated enhanced tumor growth inhibition at all dose levels tested compared to [²²⁵Ac]-Compound I monotherapy, Olaparib monotherapy, or vehicle control groups.

### Example 3. Therapeutic Efficacy of a Combination of ²²⁵Ac-Compound I and ATR Inhibitor (BAY-1895344 or Elimusertib) Treatment in the PC3-PSMA Xenograft Model

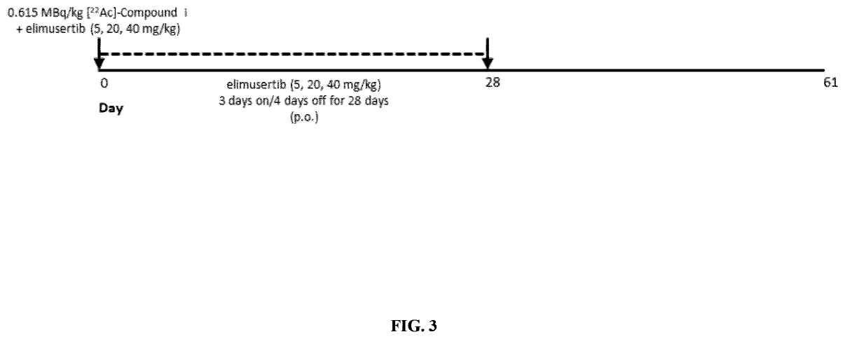

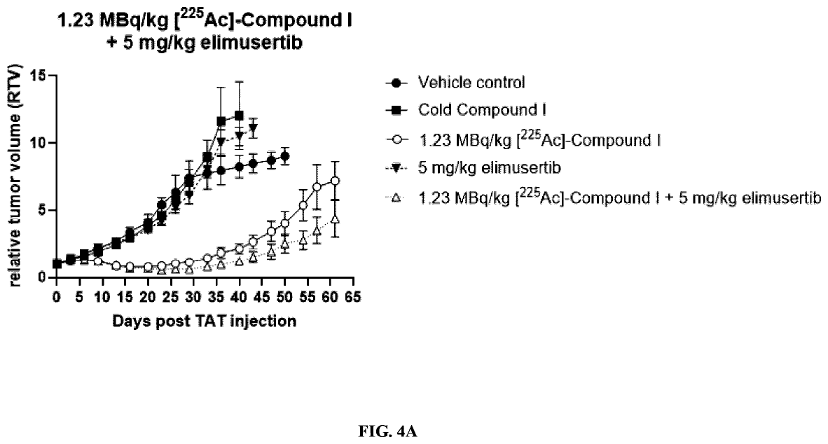

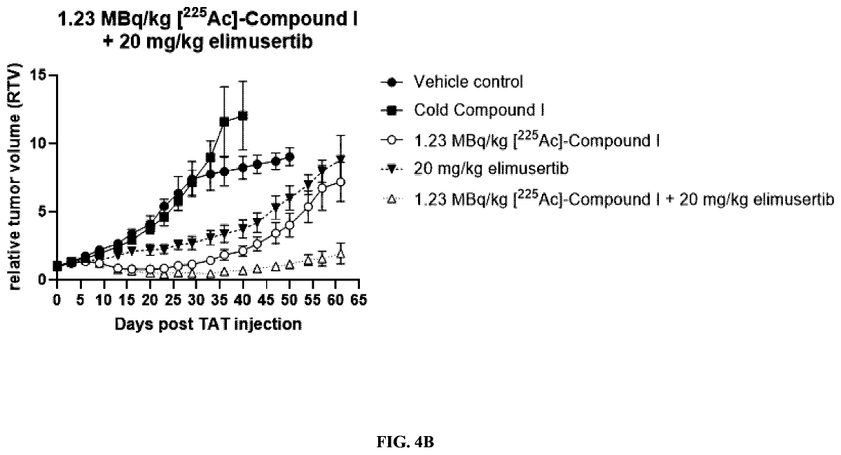

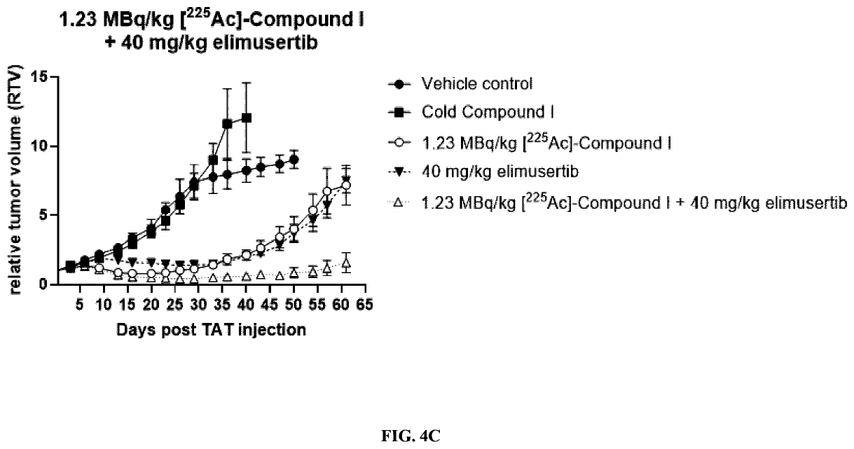

An in vivo combination study was conducted with ²²⁵Ac-Compound I and elimusertib using a PSMA overexpressing PC3 (PC3-PSMA, prostate cancer) tumor xenograft bearing mouse model. Groups of mice (n=5/group) were treated with ²²⁵Ac-Compound I, administered as a single dose (intravenous), at a dose sub-optimal for monotherapeutic efficacy (1.23 MBq/kg or 1 μCi, based on 30 g mice). Additional groups of mice (n=5/group) were dosed with elimusertib (oral gavage, twice per day) over four weekly cycles (3 days on and 4 days off), using a dose range of 5, 20, or 40 mg/kg in combination with ²²⁵Ac-Compound I. Elimusertib dosing started on the same day that ²²⁵Ac-Compound I was dosed (FIG. 3). Elimusertib monotherapy and vehicle control groups (n=5/group) were included as controls. Tumor measurements were taken 2-3 times per week and used to calculate tumor volume. Study duration was 61 days after initial therapeutic administration.

Tumor growth was tracked over time and expressed as Relative Tumor Volume (RTV; tumor volume on day X divided by initial tumor volume on day 0) (FIGS. 4A-4C). Single agent elimusertib demonstrated dose dependent efficacy as a monotherapy. Elimusertib combined with 1.23 MBq/kg of [²²⁵Ac]-Compound I, at doses of 5, 20 and 40 mg/kg, demonstrated enhanced tumor growth inhibition compared to elimusertib monotherapy, [²²⁵Ac]-Compound I monotherapy, or vehicle control groups.

### Example 4. Evaluating Therapeutic Efficacy of a Combination of ²²⁵Ac-Compound I and Olaparib (PARP Inhibitor) Treatment in Additional PSMA Models

An in vivo combination study can be conducted with ²²⁵Ac-Compound I and olaparib using another PSMA overexpressing tumor model (e.g., the spontaneous Hi-Myc PSMA model and/or a patient-derived xenograft model based on the ST1273 model) similarly as described in Example 2. (See, e.g., Simons B W et al. PSMA expression in the Hi-Myc model; extended utility of a representative model of prostate adenocarcinoma for biological insight and as a drug discovery tool. Prostate. 2019 May; 79 (6): 678-685 and. Thaysen et al., Development of a prostate cancer PDX model radioresistant to PSMA targeted radionuclide therapy. [abstract]. In: Proceedings of the American Association for Cancer Research Annual Meeting 2023; Part 1 (Regular and Invited Abstracts); 2023 Apr. 14-19; Orlando, FL. Philadelphia (PA): AACR; Cancer Res 2023; 83 (7_Suppl): Abstract nr 5039.)

Tumor growth can be monitored in monotherapy vs. combination therapy groups, and any inhibition observed can be compared between groups.

### Example 5. Evaluating Therapeutic Efficacy of a Combination of ²²⁵Ac-Compound I and ATR Inhibitor (BAY-1895344 or Elimusertib) Treatment in Additional PSMA Models

An in vivo combination study can be conducted with ²²⁵Ac-Compound I and elimusertib using another PSMA overexpressing tumor model (e.g., the spontaneous Hi-Myc PSMA model and/or a patient-derived xenograft model based on the ST1273 model) similarly as described in Example 3. (See, e.g., Simons B W et al. PSMA expression in the Hi-Myc model; extended utility of a representative model of prostate adenocarcinoma for biological insight and as a drug discovery tool. Prostate. 2019 May; 79 (6): 678-685 and. Thaysen et al., Development of a prostate cancer PDX model radioresistant to PSMA targeted radionuclide therapy. [abstract]. In: Proceedings of the American Association for Cancer Research Annual Meeting 2023; Part 1 (Regular and Invited Abstracts); 2023 Apr. 14-19; Orlando, FL. Philadelphia (PA): AACR; Cancer Res 2023; 83 (7_Suppl): Abstract nr 5039.)

Tumor growth can be monitored in monotherapy vs. combination therapy groups, and any inhibition observed can be compared between groups.

### OTHER EMBODIMENTS

Those skilled in the art will recognize or be able to ascertain using no more than routine experimentation, many equivalents to the specific embodiments described herein. Such equivalents are intended to be encompassed by the following claims.

1. A method for treating a mammal having a cancer expressing Prostate Specific Membrane Antigen (PSMA), said method comprising:
(i) administering to the mammal an ²²⁵Ac-radiopharmaceutical, wherein the mammal has received or is receiving a DNA damage response inhibitor (DDRi);
(ii) administering to the mammal a DDRi, wherein the mammal has received or is receiving an ²²⁵Ac-radiopharmaceutical; or
(iii) administering to the mammal a DDRi at the same time as administering to the mammal an ²²⁵Ac-radiopharmaceutical,
wherein in each occurrence said ²²⁵Ac-radiopharmaceutical comprises ²²⁵Ac chelated with a compound of Formula I, or a stereoisomer thereof:

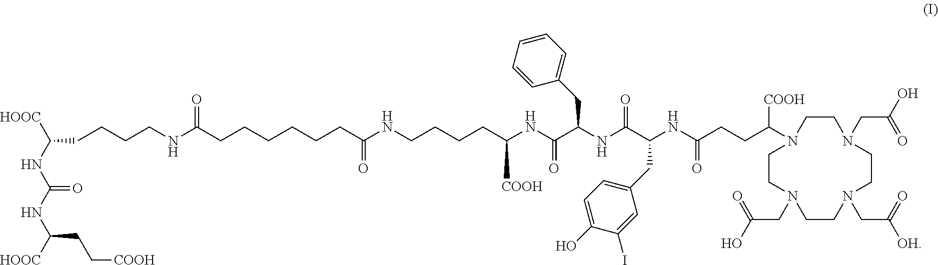 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

2. The method of claim 1, said method comprising administering to the mammal a DDRi, wherein the mammal has received or is receiving an ²²⁵Ac-radiopharmaceutical.

3. The method of claim 1 or 2, wherein said ²²⁵Ac-radiopharmaceutical comprises ²²⁵Ac chelated with a compound having the following structure:

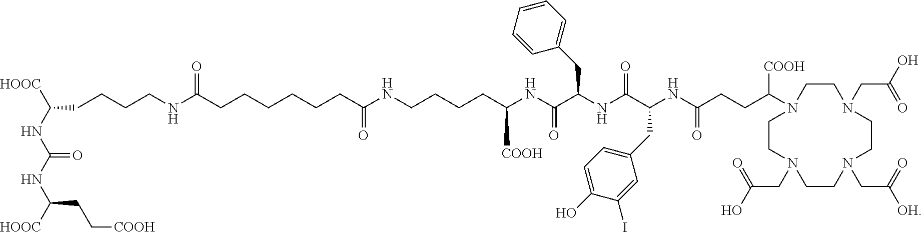 [SMILES: O=C(O)CC[C@H](NC(=O)N[C@@H](CCCCNC(=O)CCCCCCC(=O)NCCCC[C@@H](NC(=O)[C@@H](Cc1ccccc1)NC(=O)[C@@H](Cc1ccc(O)c(I)c1)NC(=O)CCC(C(=O)O)N1CCN(CC(=O)O)CCN(CC(=O)O)CCN(CC(=O)O)CC1)C(=O)O)C(=O)O)C(=O)O]

4. The method of any one of claims 1-3, wherein the DDRi is a PARP inhibitor.

5. The method of claim 4, wherein the PARP inhibitor is a small molecule PARP inhibitor.

6. The method of claim 5, wherein the small molecule PARP inhibitor is selected from the group consisting of niparib, niraparib, olaparib, saruparib, talazoparib, pamiparib, rucaparib (camsylate), and veliparib.

7. The method of claim 6, wherein the small molecule PARP inhibitor is olaparib, saruparib, or an analog thereof.

8. The method of any one of claims 1-3, wherein the DDRi is an ATR or ATM inhibitor.

9. The method of claim 8, wherein the ATR or ATM inhibitor is a small molecule ATR or ATM inhibitor.

10. The method of claim 9, wherein the small molecule ATR or ATM inhibitor is selected from the group consisting of AZ20, AZD0156, AZD1390, AZD6738, BAY-1895344 (also known as elimusertib), EPT-46464, M3541, M4344, M6620 (formerly known as VE-922 or VX-970), NU6027, and VE-821, or an analog thereof.

11. The method of claim 10, wherein the small molecule ATR or ATM inhibitor is AZD1390, BAY-1895344 (also known as elimusertib), or an analog thereof.

12. The method of any one of claims 1-3, wherein the DDRi is a DNA-protein kinase (DNA-PK) inhibitor, a WEE1 inhibitor, a Chk1 inhibitor, or a Chk2 inhibitor.

13. The method of claim 12, wherein the DDRi is a DNA-PK inhibitor selected from the group consisting of AZD7648, KU-0060648, NU7026, NU7441 (KU-57788), PI-103, PIK-75 HCI, PP121, and SF2523, or an analog thereof.

14. The method of claim 13, wherein the DNA-PK inhibitor is AZD7648 or an analog thereof.

15. The method of any one of the preceding claims, wherein the mammal is a human.

16. The method of any one of the preceding claims, wherein said ²²⁵Ac-radiopharmaceutical is administered at a dosage of less than about 2 MBq/kg of body weight of said mammal.

17. The method of any one of the preceding claims, wherein said ²²⁵Ac-radiopharmaceutical is administered at a dosage of less than about 250 kBq/kg of body weight of said mammal.

18. The method of any one of the preceding claims, wherein said ²²⁵Ac-radiopharmaceutical is administered at a dosage of less than about 100 kBq/kg of body weight of said mammal.

19. The method of any one of the preceding claims, wherein said ²²⁵Ac-radiopharmaceutical is administered as a unitary dosage of less than about 15 MBq to said mammal.

20. The method of any one of the preceding claims, wherein said ²²⁵Ac-radiopharmaceutical is administered as a unitary dosage of less than about 10 MBq to said mammal.

21. The method of any one of the preceding claims, wherein said ²²⁵Ac-radiopharmaceutical is administered as a unitary dosage of less than about 5 MBq to said mammal.

22. The method of any one of the preceding claims, wherein the cancer is selected from the group consisting of prostate cancer, breast cancer, colorectal cancer, renal cell cancer, bladder cancer, testicular-embryonal cancer, neuroendocrine cancer, and brain cancer.

23. The method of claim 22, wherein the cancer is prostate cancer or breast cancer.

24. The method of any one of the preceding claims, wherein said administering results in a decrease in tumor volume, a stable tumor volume, or a reduced rate of increase in tumor volume.

25. The method of claim 24, wherein said administering results in a decreased incidence of recurrence or decreased incidence of metastasis.

26. The method of any one of claims 1-25, wherein the mammal is administered, has received, or is receiving a lower effective dose of the ²²⁵Ac-radiopharmaceutical.
